(19) United States
(12) Patent Application Publication
(10) Pub. No.: US 2026/0225121 A1
(43) Pub. Date: Aug. 6, 2026

(54) AEROSOL DEPOSITION DEVICE

(71) Applicant: LUXEMBOURG INSTITUTE OF SCIENCE AND TECHNOLOGY (LIST), Esch-sur-Alzette (LU)

(72) Inventors: Tommaso SERCHI, Esch-sur-Alzette (LU); Sébastien CAMBIER, Esch-sur-Alzette (LU)

(21) Appl. No.: 19/142,464

(22) PCT Filed: Dec. 22, 2023
(86) PCT No.: PCT/EP2023/087532

§ 371 Date: Jun. 23, 2025

(30) Foreign Application Priority Data
Dec. 23, 2022 (EP) 22216368.5
Jan. 27, 2023 (LU) LU503396

(51) Int. Cl. B05B 13/02

(52) U.S. Cl. CPC B05B 13/0292

(57) ABSTRACT

A device (1) for applying an aerosol on a biological system includes a base and a casing, the casing having a frame (24) and four panels (36) removably inserted in the frame (24).

### TECHNICAL FIELD

The invention is directed to a device and a method for applying an aerosol to a biological system and in particular for applying an aerosol to cells maintained under air-liquid-interface conditions.

### BACKGROUND ART

Prior art patent document WO 2015/027998 A1 discloses a device for applying an aerosol on cells. The cells are placed in a container which is positioned in a stainless-steel block. An aerosol is sucked down onto the cells by a vacuum source and the aerosol is thus impacted onto the cells. Such a device requires a complex set-up and complex post-exposure operations, which prevent it from being used at a large or commercial scale. This device also results in an extremely low deposition efficiency as between only 0.1 and 1% of the aerosol reaches the cells. This device requires a long exposure time and presents a high risk of damaging the cellular layer due to the continuous exposure to the air stream.

Another example of a device for aerosol deposition is given in WO 2014/147229 A1. In this device, the inserts with the cells are inserted and taken out of the aerosol chamber one by one. This device entails many drawbacks such as a low deposition efficiency. The handling of the samples is cumbersome and this device does not propose a reliable way of deposing aerosol only on the apical side of the cells' container.

Also, these known systems do not permit the user to properly observe the level of progress of the deposition and make sure that the deposition is complete. Depending on the compound and conditions in the chamber, the aerosol may require between 5 minutes and 60 minutes to completely deposit on the samples.

### SUMMARY OF INVENTION

### Technical Problem

The invention aims at providing a device for applying an aerosol on a biological system without the drawbacks of the known devices. In particular, the invention seeks to provide a device that enables a user to correctly control and monitor the deposition process and make it more efficient and reproducible.

### Technical Solution

The invention relates to a device and a method as set out in the appended claims.

According to the invention, the device for applying an aerosol on a biological system, the device comprises: a base provided with a housing for removably receiving a multi-well plate; and a casing, removably supported by the base and delimiting an exposure chamber therein, the casing comprising a frame and four panels removably inserted in the frame.

According to an advantageous embodiment, the casing is provided with an inlet for introducing an aerosol into the exposure chamber, and the device further comprises: a multi-well plate adapted to be removably introduced in the housing of the base and having a plurality of wells configured for receiving a liquid, the wells being delimited by respective well walls; a plurality of cup-like inserts, each having a peripheral wall and a bottom in the form of a microporous membrane for receiving a biological system, each insert being adapted to be positioned in a respective well so that the peripheral wall is partially immersed in the liquid, a peripheral gap being defined between the peripheral wall of the insert and the respective well wall; and a cover adapted to lie on the base and to cover the peripheral gaps, the cover being provided with openings enabling the aerosol to flow down onto the biological system lying on each respective membrane.

According to an advantageous embodiment, at least one of the four panels is transparent, and optionally is made of glass.

According to an advantageous embodiment, the frame comprises grooves in which the panels are removably inserted.

According to an advantageous embodiment, seals are interposed between the frame and the panels, optionally in the grooves.

According to an advantageous embodiment, the frame has a top panel which exhibits four slots through which the panels can be slid into the frame.

According to an advantageous embodiment, the frame is equipped with UV LEDs.

According to an advantageous embodiment, the frame is made of metal.

According to an advantageous embodiment, the top panel is opaque and is integrally made with the frame.

According to an advantageous embodiment, the frame comprises a tab that extends parallel to a panel and that is equipped with a laser projector.

According to an advantageous embodiment, at least one of the four panels contains serigraph-printed conductors are arranged thereon.

According to an advantageous embodiment, the frame comprises a plug and the base comprises a corresponding socket, so that electric current or data can be exchanged between the base and the casing.

According to an advantageous embodiment, the frame has four corner legs that are at least three times longer than the height of the base.

The invention also relates to the casing as such for a device as noted above, the casing comprising a frame and four panels removably inserted in the frame.

The invention also relates to a method for applying a substance or an aerosol on cells with a device according to the above embodiments, the method comprising the steps of: positioning the frame of the casing on the base; then positioning the panels in the frame; and then flowing down a substance or an aerosol onto cells placed in the base, preferably on the microporous-membrane bottom of a plurality of inserts.

According to an advantageous embodiment, after the step of positioning the panels in the frame and before the step of flowing down, UV LEDs are switched ON for a given duration. The cells may be placed in the casing after this cleaning operation (for instance by opening the casing and placing the multi-well plate and the inserts in the casing).

According to an advantageous embodiment, after the step flowing down, the method comprises switching the laser projector ON, and removing the panels from the frame once the absence of droplets/cloud is observed.

Various aspects of the invention present at least the following benefits: a better handling and operation with the exposure chamber; a possibility to sterilize prior and after exposure with the UV LED mounted at the top of the frame; a possibility to visualize the aerosol droplets with the laser mounted on the side of the frame; an increased safety in operation due to hiding of electrical connections in the upper part of the frame; a possibility of replacing individual parts of the exposure box in case of rupture; a better cleaning and maintenance of the individual part of the exposure chamber; a reduced biological risk; a better control of the operations.

In the known design, the casing is made of one piece, where acrylic or polycarbonate panels are glued together. This one-piece casing is difficult to handle, clean, maintain and ultimately is fragile. Should one small breach occur, the entire part has to be replaced.

The visualization of the aerosolized cloud is not simple after the cloud is generated. Once the cloud start settling, it becomes thinner and it is difficult to understand when the sedimentation is complete. This is further complicated by the fact that the deposition time varies for each compound, being from a few minutes (5-10) to several minutes (30-60). This results in loss of chemical when the operator believes that the aerosolized cloud is completely sedimented, while, in reality, there is still some aerosol left in the chamber that is dispersed upon opening of it, resulting in lower administered dose.

The cleanness of the system could be further improved, especially for what concerns biological risk (e.g., it is not easy to sterilize the systems and rinsing with ethanol 70% is not efficient, since there are several corners and junctions in the part).

### BRIEF DESCRIPTION OF THE DRAWINGS

FIGS. 1A and 1B are respectively a cross-section and a partial top view of a schematic principle of a device for applying aerosol on a biological system;

FIG. 2 shows an exploded view of an embodiment of the device;

FIG. 3 shows an embodiment of the device in an assembled condition;

FIG. 4 shows an embodiment of the casing with heating means;

FIG. 5 shows an embodiment of the frame;

FIG. 6 shows a cross-section of a leg of the frame;

FIG. 7 shows the bottom view of the top panel of the frame;

FIG. 8 shows an exploded view and an assembled view of the device;

FIG. 9 shows a panel.

### DESCRIPTION OF AN EMBODIMENT

The drawings show some features of the invention in a schematic way. The figures are not drawn to scale and some dimensions may be enlarged for better understanding of the concepts of the invention.

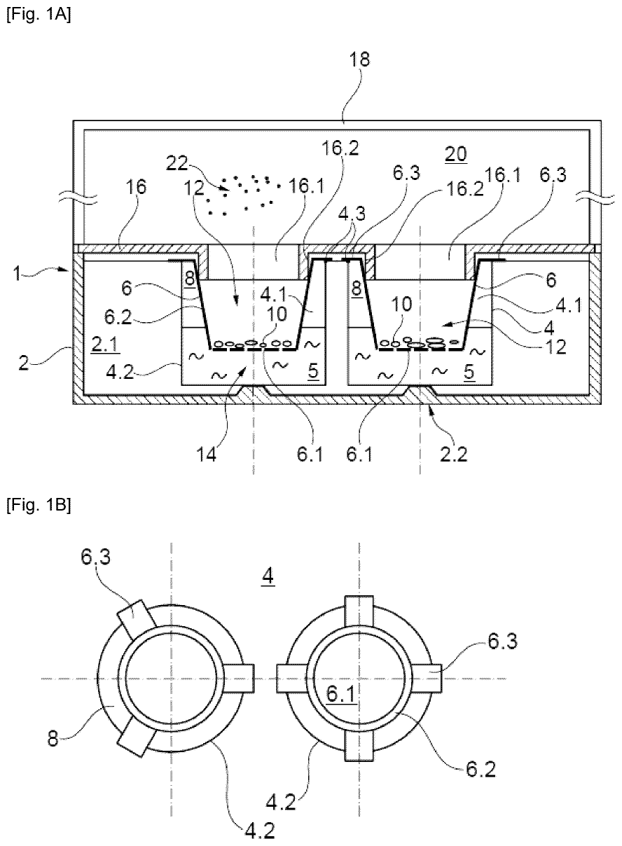

FIG. 1A shows a schematic view of part of a device for applying aerosol on a biological system.

The device 1 comprises a base 2 of a generally parallelepipedal shape and provided with a cavity or housing 2.1. At the bottom of the housing 2.1, a series of protrusions 2.2 can be arranged. The base 2 can be made of stainless steel. It can integrate heating means (e.g., a resistor embedded into the base) to heat up and/or maintain the housing at a pre-determined temperature, such as for example a temperature between 20 and 60° C., and preferably 37° C. for experiments on human cells. The base can also integrate control electronics for monitoring the temperature and an adapted interface (e.g., display) for displaying the current temperature and/or for setting a target temperature and/or a heating duration.

Integrating the heating means within the base enables a biologically and chemically non-intrusive heating and temperature monitoring while preventing the need for an external heating which would require handling time.

A multi-well plate 4 can be inserted in the housing 2.1, for instance laying on the protrusions 2.2. The plate 4 may be a standard cell culture plate, containing a plurality of wells 4.1 (e.g., 4, 6, 12, 24, etc.). The wells 4.1 can be filled independently with liquid. The wells 4.1 are delimited by well walls 4.2 which can have a generally cylindrical shape. A generally circular edge 4.3 constitutes the top end of the well walls 4.2.

In each well, an insert 6 can be introduced. The insert 6 can have a generally cup-like shape. The insert 6 has a microporous membrane 6.1 as bottom (for example with pores comprised between 2 and 10 pm) and has a peripheral wall 6.2 which can be cylindrical or conical. At the top of the peripheral wall 6.2, a ring (not shown) or alternatively fingers 6.3 can be arranged, so that the insert 6 hangs in the well, supported by the circular edge 4.3.

The peripheral wall 6.2 and the well wall 4.2 can have any other regular or irregular geometric cross-section (e.g., polygon, etc.).

A gap 8 between the peripheral wall 6.2 and the well wall 4.2 facilitates the introduction and removal of the insert 6 into/from the well 4.1 (for instance with a tweezer). Various sizes of inserts 6 can be used with the same well plate 4. The inserts 6 may be loosely engaged in the respective wells 4.1. This again enables a simple and fast introduction of the inserts in the well. It also allows various sizes of inserts to be used with the same multi-well plate (simultaneously or sequentially).

FIG. 1B shows a partial top view of the device 1, illustrating two exemplary inserts 6 in their respective wells 4.1. Various numbers of fingers 6.3 can be foreseen. Between the well wall 4.2 and the inserts' walls 6.2 lies the gap 8.

Back in reference to FIG. 1A, cells 10 can be deposited on the membrane 6.1. An apical compartment 12 stands above the cells 10 and a basolateral compartment 14 lies below and around the insert 6. The basolateral compartment 14 is maintained immersed in the liquid 5, while the apical compartment 12 is not immersed.

To maintain the apical compartment 12 sealed away from the basolateral compartment 14, a cover 16 is provided. The cover 16 lays on the base 2 and/or on the multi-well plate 4 and/or on the inserts 6. The cover 16 has a generally plate-like shape with a plurality of openings 16.1. The cover 16 covers the gaps 8 and can be provided with protrusions 16.2 which extend downwards into contact with the inserts 6. The outline of the protrusions 16.2 matches the outline of the peripheral wall 6.2 of the inserts 6, hence the protrusions 16.2 may be generally circular. The protrusions 16.2 help to seal the apical compartment 12 from the basolateral compartment 14. The protrusions 16.2 also help centring the inserts 6 with respect to the respective opening 16.1. This facilitates the set up and centring of the inserts in the wells as they do not need to be manually centred. The inserts are indeed aligned all at once when positioning the cover down on the inserts. This quickens the handling of the inserts and improves both the efficiency and reliability of the whole process of deposition, by facilitating the handling and ensuring a proper positioning of the cells.

The cover 16 may be made of a biocompatible material so as to be disposable (e.g., biocompatible polymer such as polyethylene, ABS, polycarbonate, etc.). The cover 16 may be chemically resistant and autoclavable. The cover may be manufactured by injection moulding to limit the costs involved and facilitate mass production. By providing such a disposable cover, the cross-contamination between two successive experiments is prevented and the need to clean the cover is waived, rendering the deposition process more reliable and more efficient.

The cover 16, the multi-well plate 4 and the inserts 6 are removable as a group from the base. This enables to leave the inserts in the wells and to carry them as a group to the post-exposure incubation, thereby preventing cross-contamination (as would be the case when removing each insert one after the other). Also, this enables to prepare an experiment by inserting the inserts in a multi-well plate and sealing the wells with a cover, aside from the device, while another multi-well plate and other inserts are being processed in the chamber, improving thereby both reliability of the experiments and time efficiency.

Above the base 2, a 5-sides box-like casing 18 can be arranged. The casing 18 can be made of any material, as for example, polycarbonate, plexiglass, glass, acrylic or polypropylene. The casing 18 delimits a cavity therein, i.e., the exposure chamber 20. An aerosol 22 can be formed in the exposure chamber 20. The aerosol 22 may be of any nature. For example, a substance that is to be tested on the cells 10 can be diluted in water solution with salt. A nebulizer (not shown), e.g., a high-voltage membrane arranged horizontally, can nebulize the solution to form a thick cloud that distributes evenly in the exposure chamber 20, before settling down onto the cells 10. Various technologies for the generation of the aerosol can be envisaged, such as those well-detailed in WO 2014/147229 A1.

The openings 16.1 allow the aerosol 22 to flow down onto the cells 10, while the design of the cover 16, of the inserts 6 and of the multi-well plate 4 prevent the aerosol 22 from reaching the basolateral compartment 14.

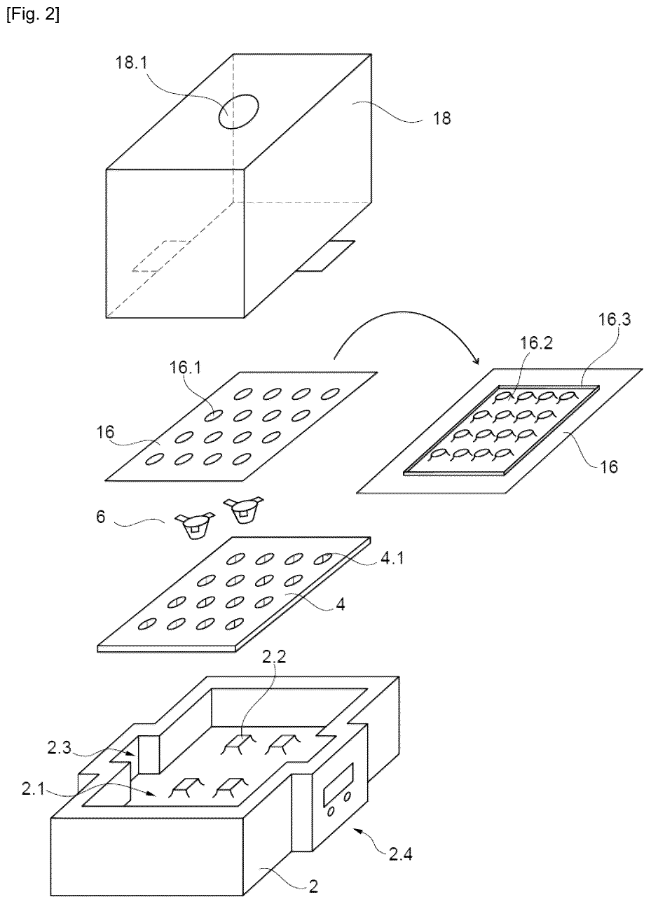

FIG. 2 illustrates an exploded view of the various parts of an embodiment of the device 1. The same numbers as in FIG. 1 are used to depict the same parts.

One can see that the base 2 comprises two lateral extensions 2.3 which can form a recess between the plate 4 and the base 2. These recesses enable an easy insertion and/or removal of the plate 4 by hand. The base 2 can furthermore have ribs or chamfers to adequately position the plate 4 laterally (in the horizontal plane). A user interface 2.4 (display, buttons, alarm, etc.) can be provided, preferably on one of the extensions 2.3. This interface 2.4 is connected to electronics controlling the temperature of the base 2 through sensors and heating means.

FIG. 2 also shows the cover 16 with the protrusions 16.2 (upside down view on the right-hand side) and a peripheral lip 16.3 which can enhance the sealing of the aerosol away from the multi-well plate 4, or the positioning of the cover 16: the plate 4 can be in contact with the inner side of the lip 16.3 and/or the base 2 can be in contact with the outer side of the lip 16.3.

The casing 18 exhibits an opening 18.1, preferably at the top of the casing 18, serving as an inlet for the aerosol.

The casing 18 may be made of a top side where the opening 18.1 is arranged, and four lateral sides. The casing 18 and its sides, or at least one of them, may be transparent.

The casing 18 has a height that is at least 3 times, and preferably at least 5 times the height of the base.

Alternatively, or in combination, the casing may be at least 10 cm high, while the base is less than 8 cm high.

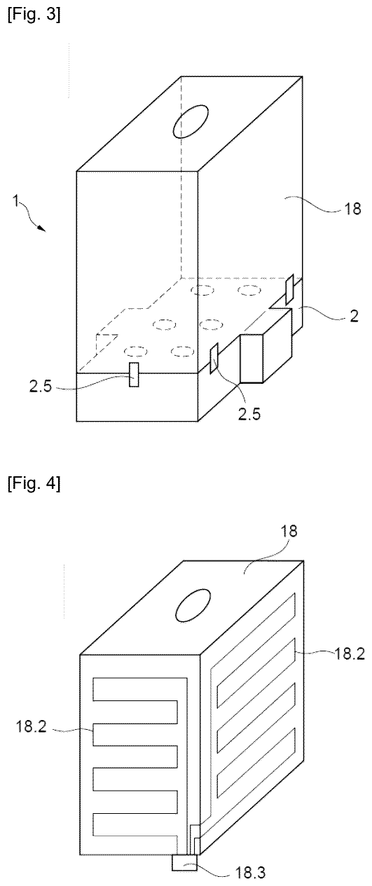

FIG. 3 shows the device in an assembled condition. This figure shows elastic tabs 2.5, fixed to the base 2 and adapted for a snap-fit engagement with the casing 18. Other assembling design can also be provided (flanges, screws, quick-lock, etc.). Those aspects constitute further improvements for handling the device more efficiently.

FIG. 4 shows an embodiment wherein the casing 18 comprises heating means 18.2.

In one embodiment, the heating means 18.2 can be in the form of silk-screen printed (serigraph-printed) conductors. To feed the heating means 18.2 with power, an electric plug 18.3 can engage a socket in the base 2.

The conductor 18.2 may have a pattern with regular crenels extending over one or more of the sides of the casing 18. Alternatively, an irregular pattern can be used for carrying out experiments where it is desired to have a heterogenous temperature in the exposure chamber.

The heating means 18.2 may be arranged on the lateral sides of the casing which may be transparent.

In another embodiment (not shown), electric resistors can serve as heating means. They can be arranged as an array over at least one side of the casing 18 or can be embedded in housings formed in the casing's walls.

In yet another embodiment, the casing can be provided with a network of pipes (e.g., manufactured by additive manufacturing), so as to enable a fluid to heat the walls. The fluid may be heated by external means or by the heating means of the base 2, through a heat exchanger. Appropriate pumping and control means can be provided.

Additionally, fans can be arranged around the walls of the casing 18 to reduce the casing's temperature if needed, hence more precisely controlling its temperature.

Control means can be provided to maintain the temperature in the exposure chamber 20 at a desired temperature, for instance a temperature between 20 and 60° C., and preferably between 36 and 38° C. The control electronics can be integrated with the control means of the temperature of the base 2. Hence, the interface 2.4 of the base 2 can allow independent or common control of the temperature of the base 2 and of the casing 18.

The heating means 18.2 improve the efficiency of the deposition as they prevent condensation of the aerosol on the walls of the casing 18, ensuring thereby that 3 to 5 times more of aerosol is deposited than in existing devices.

Indeed, when the casing 18 is not heated, the aerosol could condensate on the walls of the casing.

The absence of condensation leads to an enhanced deposition rate and allows a better visual control of the process, since the walls are not clouded by condensation.

The present invention can be used in an in vitro model of a human lung for assessing the irritation potential, the sensitization or toxicity of inhalable products such as particles or molecules on the alveolar barrier of lungs (see e.g., WO 2018/122219 A1). For that purpose, epithelial cells and macrophages can be present at the apical side of the membrane, and endothelial cells and dendritic-like cells are present at the basal side of the membrane, immersed in a co-culture medium.

The device and method can however be used for any other application where an aerosol is to be deposited efficiently and reliably on a biological system.

A comparative example is presented hereinafter.

### Objective

The aim of this experiment was to evaluate the effect of the casing temperature on its deposition efficiency. A comparative study to quantify the deposited sodium fluorescein (used as reference compound) by using a system A (with housing temperature set at 37° C.) and a system B (with both housing and casing at 37° C.) was performed.

### Materials Needed

Fluorescein sodium salt in powder form (CAS No.: 518-47-8)
Dulbecco's Phosphate Buffered Saline (e.g. GIBCO™ DPBS, no calcium, no magnesium—Cat. No.: 14190144 or 14190094, ThermoFisher)
Distilled water
Two exposure systems with Aerogen Nebulizer, “system A” without heating means, “system B” with heating means for controlling the temperature of the casing
Transwell inserts (1 μm pore size—Millipore-Merk)
Fluorescence plate reader (e.g. Tecan SparkControl)
Vortex shaker
Micropipettes 20-200 μL, 100-1000 μL with corresponding tips
Eppendorf tubes (1.5 mL)
1 Falcon tube (15 mL)
Flat bottom, black 96-well multi-well plate (e.g. Greiner CELLSTAR® 96 well plate black polystyrene flat bottom)
Aluminium foil

### General Notes

Fluorescein sodium salt solution needs to be shielded from light in order to prevent photo-bleaching.
Fluorescein sodium salt solutions need to be pH-buffered, since its fluorescence efficiency strongly depends on pH. Hence, DPBS is used here as solvent.

### Experimental Procedure

1. Clean both systems
   a. Clean both exposure systems with clear ethanol 70° (stainless steel module as well as exposure chamber).
2. Prepare the fluorescein solution for nebulization
   a. Prepare a 15 μg/mL fluorescein (stock) solution in DPBS and store it in a falcon tube wrapped with aluminium foil.
   b. Prepare five fluorescein solutions with lower concentrations for a standard curve. Dilute the 15 μg/mL fluorescein stock solution with DPBS to 0.375 μg/mL (in DPBS) and generate a 1:2 dilution series (0.3750 μg/mL, 0.1875 μg/mL, 0.0938 μg/mL, 0.0469 μg/mL, 0.0234 μg/mL, blank DPBS).
      These standard solutions are stable in the dark for around 1 h.
3. Set the temperature controller of the system A to 37° C. and allow ample time for warm-up (ca. 20 min). While the system is warming up the following steps (number 5 below) can be performed.
4. Set the temperature controller of the system B to 37° C. (both top, casing, and bottom, housing) and allow ample time for warm-up (ca. 20 min). While the system is warming up the following steps (number 5 below) can be performed.
5. Preparation of experiments with systems A and B:
   a. Insert a 6 MW plate inside the stainless-steel scaffold module removing the original lid.
   b. Fill each insert with 800 μL of DPBS carefully checking to homogeneously distribute the liquid onto the surface of the insert. Note 1: The DPBS in the insert collects the aerosolized fluorescein solution depositing on the bottom of the insert, where normally the cells are residing.
   c. Place the 500 HP pierced lid on the top of the plate and fill wells 1, 3, 5 with Millipore Transwell inserts (1 μL pore size).
   d. Close the system with the glass casing and place on its top the nebuliser previously cleaned by spraying 500 μL of DPBS under the laminar flow hood equipped with HEPA filter.
6. Sodium Fluorescein Nebulization
   Pipette 200 μL of 15 μg/mL fluorescein stock solution on each nebuliser, close the cap and start the aerosolization by connecting the nebuliser to the electricity. After the formation of the cloud leave for additional 5 minutes the casing on.
7. Measuring fluorescence intensity
   a. Remove the casing from the devices and pipette an aliquot of 300 μL from the centre of each Transwell insert into different Eppendorf tubes. Note 1: Do not shake or tilt the inserts to avoid inadvertent washing of aerosol from the walls of the inserts. Note 2: Mix the solution in the liquid prior to withdrawal from insert e.g. by taking 200 μL from the edge of the insert and dropping it to the centre carefully 2 or 3 times. Note 3: Protect the tubes from light.
   b. Vortex the tubes and load 100 μL of sample solutions and Sodium Fluorescein standard solutions in a black 96 MW plate.
   c. Measure the fluorescence signals for each solution 96-well plate with a fluorescence reader. (excitation/emission: 483/525 nm; bandwidth 10 nm)
   d. Clean the nebuliser between consecutive exposures by spraying 200 μL of DPBS under the laminar flow hood equipped with HEPA filter.
   e. Clean the internal walls of the exposure chamber by whipping away the aerosol that could be left.
   f. Repeat the whole procedure for 3 times.

### Results

The deposited dose of Sodium Fluorescein was calculated based on the standard curve (acquisition of the standard solutions OD was done for each single measurement). The obtained values (μg/mL) were then normalised on the average of deposited fluorescein measured when using the system A at 37° C.
Thus, the results are expressed as fold increase of the estimated concentration of sodium fluorescein deposited on the Transwell inserts by using the system B (both housing and casing at 37° C.) compared to the deposition obtained by using the system A (only housing at 37° C.).
Table 1 summarises the results obtained for the two systems.

TABLE 1
Fluorescence intensity fold increase
compare to system A (at 37° C.)

| | System A | System B |
|---|---|---|
| | (Fold increases | (Fold increases |
| | compare to A) | compare to A) |
| Experimentation 1 | 0.9 | 5.0 |
| Experimentation 2 | 1.0 | 5.0 |
| Experimentation 3 | 1.1 | 4.0 |
| Average Fl | 1.0 | 4.8 |
| SD | 0.1 | 0.4 |

The deposition efficiency observed for the system B is 4 to 5 times higher than the one observed using system A.
These experimentations show that the difference is to be attributed to the double heating of the system B. This system is equipped with specific heating means (resistors and silk-screen printed casing) allowing for the independent heating of both the components (housing and casing). The conductive serigraphy of the glass chamber keeps the temperature quite constant on the top of the system reducing the deposition of aerosols on the walls of the casing itself. During the nebulization step it is possible to observe the formation of the cloud which is continuously re-circulating within the exposure chamber and no condensation on the casing walls was observed.
On the contrary, when the nebulization is performed using the system A, the aerosol is partially condensing on the walls of the device, most probably reducing the amount of aerosol available for the deposition on the biological system.
Similar experimentations at various other temperatures from 20 to 80° C. are expected to show a similar improvement of system B over system A.
Also, experimentations with other aerosols are expected to result in the same improvement of system B over system A.

According to the invention, the casing 18 is made of a frame in which panels are removably inserted.

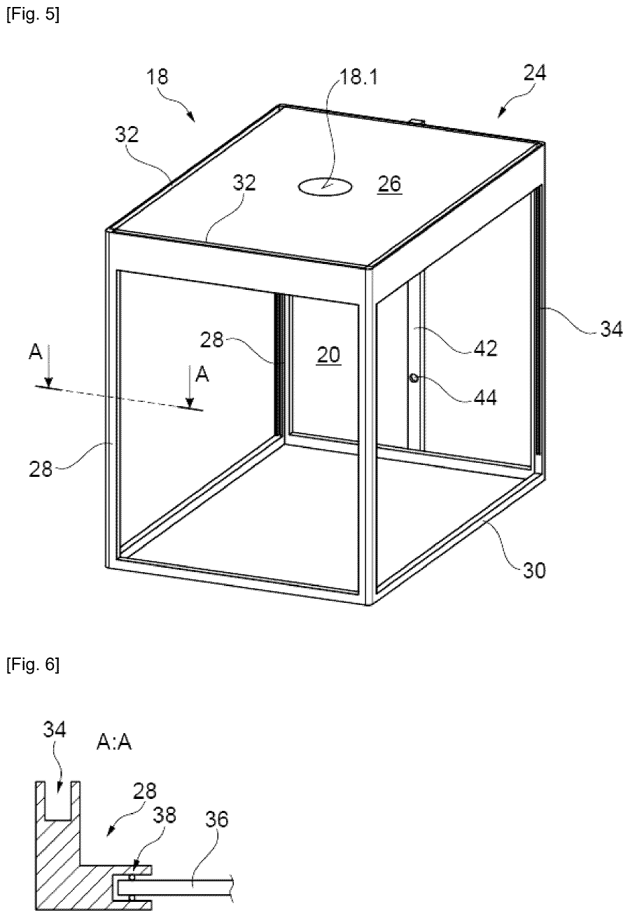

FIG. 5 shows an exemplary embodiment of the frame 24. The frame may be made of metal and may be coated or treated to render the frame inert to the intended biological experimentations. The frame may have a top panel 26 and four legs 28 extending parallel to one another.

At least one of the legs 28 can contain the cables and the plug (18.3 on FIG. 4) that may be necessary to receive power and/or to transmit/receive data to/from the base 2.

The feet of the legs 28 can be connected to one another with connecting tabs 30, to make the casing rigid and/or to help sealing the chamber.

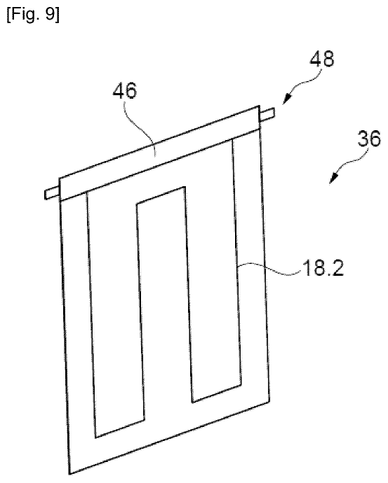

In one embodiment, the top of the frame can have slots 32 that enable the glass panels (see also FIG. 9) to be inserted into the frame from above, i.e., rendering the insertion even possible when the frame is already positioned on the base. Alternatively, in an embodiment without slots 32, the panels can be inserted from below.

The window defined on each side between the legs 28, the tab 30 and the top panel 26 may constitute observation openings for a user to observe the chamber 20 through transparent panels. The area of the window may be at least a quarter of the area of a side of the casing 18.

FIG. 5 also illustrates a tab 42 which extends in the middle of one side of the frame 24. This tab 42 is equipped with a laser source 44 that is oriented to project laser light (e.g., green) in the chamber. This enables the operator to observe more accurately whether the cloud or aerosol is still in suspension in the chamber.

The laser 44 may alternatively be positioned somewhere else in the frame, i.e., on a leg 28 or the top panel 26. Also, the tab 42 may have any other shape or orientation to enable the laser 44 to illuminate a particularly interesting region of the chamber 20.

FIG. 6 shows a cross-section of a leg 28 of the frame. The legs 28 may be provided with grooves 34 to receive the glass panels 36. The width of the groove 34 may match the thickness of the panel 36. Alternatively, a seal 38 on one side and/or on the other side of the panel 36 can be foreseen. The seal may be made of an inert elastic material (e.g., elastomer).

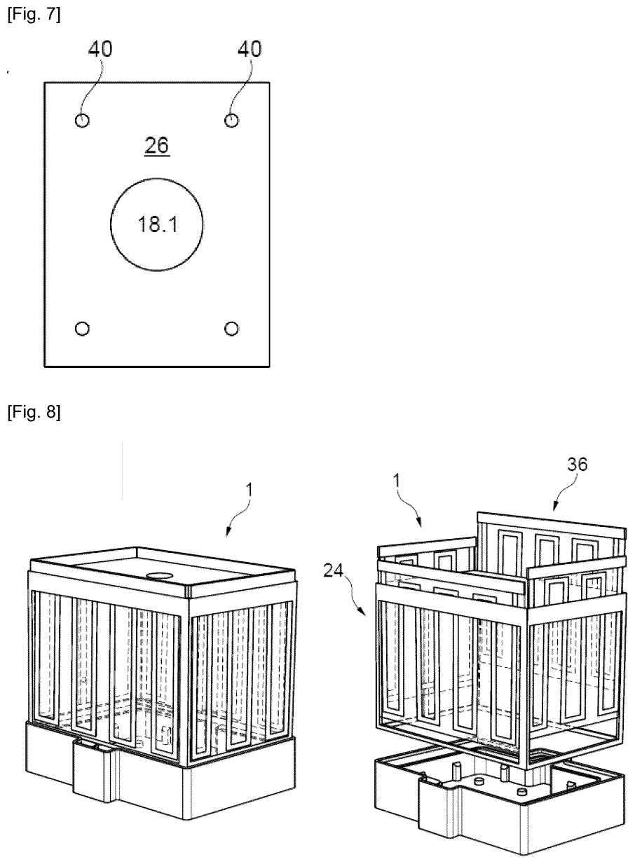

FIG. 7 shows the bottom view of the top panel 26 of the frame 24, i.e., seen from within the chamber 20. The top panel may be provided with one or more UV LEDs, arranged in an array. These LEDs enable to sterilize the device and/or the casing, before and/or after use.

The electric cables and/or the electronics necessary for feeding the LEDs with power can be hidden within the top panel 26. A maintenance hatch can be foreseen on the top surface of the top panel, to allow maintenance (e.g., replacing a battery) even during use.

FIG. 8 shows an assembled view and an exploded view of the device. In this example, the panels 36 are inserted into the frame 24 from above.

FIG. 9 shows a panel 36. The panel 36 may be transparent and may be made of glass. It may have a serigraph-printed conductor 18.2. The top of the panel 46 can be a small rod made of metal or plastic and provided for handling the panel 36 safely and hygienically. The rod 46 can have two ends 48 provided with electric connectors to feed the conductor 18.2 with power.

The casing may contain at least one transparent panel, while the other panels may be opaque. The non-transparent panels may be integral with the frame. The top panel 26 may also be transparent and/or may also be made of a separate piece that can be fixed removably to the frame 24.

The device enables to carry out a method wherein the frame is arranged on the base first, and only then the panels are inserted into the frame, before flowing down a substance on the cells. UV lights can be used before and/or after the experimentations to clean the frame and/or the base and/or the panels. The laser can be switched ON to check whether any droplet remains in suspension before opening the casing after an experimentation.

The method may also comprise: placing cells on the microporous membrane-like bottom of a plurality of inserts; placing the inserts in respective wells of a multi-well plate, thereby defining an apical compartment and a basolateral compartment, partially immersed in liquid; covering the inserts and the multi-well plate with a cover, the cover being provided with openings exposing the apical compartment of the inserts while sealing away the basolateral compartment; and introducing the multi-well plate, the inserts and the cover in the housing of the base and positioning the casing on the base to seal the exposure chamber.

1. A device for applying an aerosol on a biological system, the device (1) comprising:
a base provided with a housing for removably receiving a multi-well plate; and
a casing, removably supported by the base and delimiting an exposure chamber therein, the casing comprising a frame and four panels removably inserted in the frame.

2. The device according to claim 1, wherein the casing is provided with an inlet for introducing an aerosol into the exposure chamber, and the device further comprises:
a multi-well plate adapted to be removably introduced in the housing of the base and having a plurality of wells configured for receiving a liquid, the wells being delimited by respective well walls;
a plurality of cup-like inserts, each having a peripheral wall and a bottom in the form of a microporous membrane for receiving a biological system, each insert being adapted to be positioned in a respective well so that the peripheral wall is partially immersed in the liquid, a peripheral gap being defined between the peripheral wall of the insert and the respective well wall; and
a cover adapted to lie on the base and to cover the peripheral gaps, the cover being provided with openings enabling the aerosol to flow down onto the biological system lying on each respective membrane.

3. The device according to claim 1, wherein at least one of the four panels is transparent.

4. The device according to claim 1, wherein the frame comprises grooves in which the panels are removably inserted.

5. The device according to claim 1, wherein seals are interposed between the frame and the panels.

6. The device according to claim 1, wherein the frame has a top panel which exhibits four slots through which the panels can be slid into the frame.

7. The device according to claim 1, wherein the frame is equipped with UV LEDs.

8. The device according to claim 1, wherein the frame is made of metal.

9. The device according to claim 1 wherein the top panel is opaque and is integrally made with the frame.

10. The device according to claim 1, wherein the frame comprises a tab that extends parallel to a panel and that is equipped with a laser projector.

11. The device according to claim 1, wherein at least one of the four panels contains serigraph-printed conductors are arranged thereon.

12. The device according to claim 1, wherein the frame comprises a plug and the base comprises a corresponding socket, so that electric current or data can be exchanged between the base and the casing.

13. The device according to claim 1, wherein the frame has four corner legs that are at least three times longer than the height of the base.

14. A casing for a device according to claim 1, the casing comprising a frame and four panels removably inserted in the frame.

15. A method for applying a substance or an aerosol on cells with the device in accordance with claim 1, the method comprising the steps of:
positioning the frame of the casing on the base; then
positioning the panels in the frame; and then
flowing down a substance or an aerosol onto cells placed in the base.

16. The method according to claim 15, wherein after the step of positioning the panels in the frame and before the step of flowing down, UV LEDs are switched ON for a given duration.

17. The method according to claim 15, wherein said method further comprises, after the step flowing down, switching the laser projector ON, and removing the panels from the frame once the absence of droplets/cloud is observed.
